(19) United States
(12) Patent Application Publication
(10) Pub. No.: US 2006/0259128 A1
(43) Pub. Date: Nov. 16, 2006

(54) Method for implanting prosthetic valves

(75) Inventors: Dusan Pavcnik, Portland, OR (US); Brian C. Case, Bloomington, IN (US); Ram H. Paul JR., Bloomington, IN (US); Jacob A. Flagle, Indianapolis, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/404,439

(22) Filed: Apr. 14, 2006

Related U.S. Application Data
(60) Provisional application No. 60/672,440, filed on Apr. 18, 2005.

(51) Int. Cl. A61F 2/06

(52) U.S. Cl. 623/1.24; 623/903

(57) ABSTRACT

Methods for implanting a prosthetic valve in a body vessel having a fluid flow therethrough are provided. One method includes identifying a position of an existing valve and determining a factor affecting fluid flow in at least one of a first direction and a second direction at the existing valve. The method includes selecting the implantation position for a prosthetic valve at a distance away from the existing valve position in consideration of the factor. The method further includes providing the prosthetic valve for delivery to the implantation position, delivering and implanting the prosthetic valve at the position. The prosthetic valve includes at least one flexible member movable between a first position that permits fluid flow in the first direction and the second position that substantially prevents fluid flow in a second direction.

### RELATED APPLICATIONS

This application claims the benefit of U.S. Provisional Application No. 60/672,440, filed Apr. 18, 2005, which is incorporated by reference herein in its entirety.

### TECHNICAL FIELD

The present invention relates to medical devices. More particularly, the invention relates to methods for implantation of an intraluminal device in a body vessel.

### BACKGROUND OF THE INVENTION

Many vessels in animals transport fluids from one bodily location to another. Frequently, fluid flows in a substantially unidirectional manner along the length of the vessel. For example, veins in the body transport blood to the heart and arteries carry blood away from the heart.

In mammalian veins, natural valves are positioned along the length of the vessel in the form of leaflets disposed annularly along the inside wall of the vein which open to permit blood flow toward the heart and close to restrict back flow. These natural venous valves open to permit the flow of fluid in the desired direction and close upon a change in pressure or when muscles relax or stop contraction. When blood flows through the vein, the pressure forces the valve leaflets apart as they flex in the direction of blood flow and move towards the inside wall of the vessel, creating an opening therebetween for blood flow. When the pressure differential across the valve, the flow velocity, or both change, the leaflets return to a closed position to restrict or prevent blood flow in the opposite, i.e. retrograde, direction. The leaflet structures, when functioning properly, extend radially inwardly toward one another such that the tips contact each other to restrict backflow of blood.

In the condition of venous insufficiency, the valve leaflets do not function properly. Incompetent venous valves can result in symptoms such as swelling and varicose veins, causing great discomfort and pain to the patient. If left untreated, venous insufficiency can result in excessive retrograde blood flow through incompetent venous valves, which can cause venous stasis ulcers of the skin, pain and discoloration.

There generally are two types of venous insufficiency: primary and secondary. Primary venous insufficiency typically occurs where the valve structure remains intact, but the vein is simply too large in relation to the leaflets so that the leaflets cannot come into adequate contact to prevent backflow. More common is secondary venous insufficiency, where the valve structure is damaged, for example, by clots which gel and scar, thereby changing the configuration of the leaflets, i.e. thickening the leaflets and creating a “stub-like” configuration. Venous insufficiency can occur in the superficial venous system, such as the saphenous veins in the leg, or in the deep venous system, such as the femoral and popliteal veins extending along the back of the knee to the groin.

A common method of treatment of venous insufficiency is placement of an elastic stocking around the patient's leg to apply external pressure to the vein. Although sometimes successful, the tight stocking is quite uncomfortable, especially in warm weather, as the stocking must be constantly worn to keep the leaflets in apposition. The elastic stocking also affects the patient's physical appearance, thereby potentially having an adverse psychological affect. This physical and/or psychological discomfort can lead to the patient removing the stocking, thereby preventing adequate treatment.

Surgical methods for treatment of venous insufficiency have also been developed. A vein with incompetent venous valves can be surgically constricted to bring incompetent leaflets into closer proximity in an attempt to restore natural valve function. Methods for surgical constriction of an incompetent vein include implanting a frame around the outside of the vessel, placing a constricting suture around the vessel, or other types of treatment of the outside of the vessel to induce vessel contraction. Other surgical venous insufficiency treatment methods include bypassing or replacing damaged venous valves with autologous sections of veins with competent valves. However, these surgeries often result in a long patient recovery time and scarring, and carry the risks, e.g. anesthesia, inherent with surgery.

Recently, various implantable prosthetic devices and minimally invasive methods for implantation of these devices have been developed to treat venous insufficiency, without the disadvantages of treatment with an outer stocking or surgery. Such prosthetic venous valve devices can be inserted intravascularly, for example from an implantation catheter. Prosthetic devices can function as a replacement venous valve, or restore native venous valve function by bringing incompetent valve leaflets into closer proximity.

Some intraluminal medical devices include a functional mechanism that is sensitive to positioning within the body vessel. In addition, positioning of the prosthetic valve with respect to an existing valve may improve the function of the prosthetic valve, the existing valve, or both. What is needed in the art are methods for delivering prosthetic valve devices to a desired position at a distance away from the existing valve within the body vessel.

The present invention provides various methods for improving valve function utilizing a prosthetic valve device and the positioning of the prosthetic valve device with respect to an existing valve.

### BRIEF SUMMARY OF THE INVENTION

In one embodiment of the present invention, a method for implanting a prosthetic valve in a body vessel having a fluid flow therethrough is provided. The method includes identifying a position of an existing valve and determining a factor affecting fluid flow in at least one of a first direction and a second direction at the existing valve. The method includes selecting the implantation position for a prosthetic valve at a distance away from the existing valve position in consideration of the factor. The method further includes providing the prosthetic valve for delivery to the implantation position, delivering and implanting the prosthetic valve at the position. The prosthetic valve includes at least one flexible member movable between a first position that permits fluid flow in the first direction and the second position that substantially prevents fluid flow in a second direction.

In another embodiment of the present invention, a method for modulating fluid flow through a body vessel is provided. The method includes identifying an existing incompetent valve in the body vessel where the existing valve allows fluid flow therethrough in a first direction and a second direction. The method further includes determining an implantation site for placement of a prosthetic valve at a distance away from the existing valve, providing the prosthetic valve, delivering the prosthetic valve to the implantation site and implanting the valve at the implantation site. Implanting the prosthetic valve at the distance modifies fluid flow through the body vessel by substantially preventing fluid flow in the second direction and substantially maintaining flow vortices formed at the existing valve.

In yet another embodiment of the present invention, a method of implanting a prosthetic valve in a body vessel is provided. The method includes identifying a first axis of an opening in an existing valve in the body vessel and determining a factor affecting fluid flow in at least one of a first direction and a second direction at the existing valve. The method includes selecting an implantation position for a prosthetic valve at a distance away from the existing valve in consideration of the factor. The method further includes providing the prosthetic valve having an opening having a second axis, delivering the prosthetic valve to the implantation position in the body vessel, positioning the second axis with respect to the first axis and implanting the prosthetic valve in the body vessel with the second axis positioned with respect to the first axis and at the distance away from the existing valve.

Advantages of the present invention will become more apparent to those skilled in the art from the following description of the preferred embodiments of the invention which have been shown and described by way of illustration. As will be realized, the invention is capable of other and different embodiments, and its details are capable of modification in various respects. Accordingly, the drawings and description are to be regarded as illustrative in nature and not as restrictive.

### BRIEF DESCRIPTION OF SEVERAL VIEWS OF THE DRAWINGS

FIG. 1 is a cross-sectional view of a native venous valve and blood flow pattern;

FIG. 2A is a side view of a vessel showing an embodiment of the implanted prosthetic valve in an open position according to the present invention;

FIG. 2B is a side view of the vessel shown in FIG. 2A showing the prosthetic valve in a closed position according to the present invention;

FIG. 3 is a side view of an embodiment of the implanted prosthetic valve of the present invention in an alternative placement compared to FIGS. 2A and 2B;

FIG. 4 is a cross-section view of an embodiment of the present invention showing a prosthetic valve positioned with respect to an existing valve;

FIG. 5 is a cross-sectional view of an alternative embodiment of the valve position shown in FIG. 4;

FIG. 6 is a cross-sectional view of an alternative embodiment showing an alternative position;

FIG. 7 is a side view of an embodiment of the present invention showing an imageable element on the prosthetic valve; and

FIG. 8 is a side view of a delivery device for placing a valve in position.

### DETAILED DESCRIPTION OF THE INVENTION

As described herein, a method is provided for delivering a prosthetic valve to a predetermined position within a body site having fluid flow therethrough and having an incompetent valve. The valves of the present invention are suitable for implantation into vessels. The term vessel as used herein includes ducts, canals, and other passageways in the body, as well as cavities and other locations. For example, the valves of the present invention are suitable for implantation into the vessels of the vasculature, such as veins, for regulating fluid flow through the vessel. The valves of the present invention may also be implanted in a passageway of the heart to regulate the fluid flow into and out of the heart. The incompetent valve may be an existing natural valve or an existing prosthetic valve. The prosthetic valve of the present invention may be placed within a vessel such that any retrograde flow vortices formed at the incompetent valve continue to form to flush out fluids collecting within sinuses at the existing valve after the prosthetic valve is positioned at the predetermined position. The vortices formed at a native competent valve are illustrated in FIG. 1. A native competent valve 1 is shown wherein retrograde blood 2 flowing back toward the valve 1 and closing the valve 1 creates a series of vortices 3 as the retrograde flow 2 contacts leaflets 4 and 5. Rounded sinuses 6 that are naturally formed at the valve 1 are thought to facilitate the creation of the vortices 3. The vortices 3 help prevent blood from pooling or stagnating within pockets 7 formed at the base of the valve 1. Stagnation of the blood may lead to thrombus formation or other problems. Although the existing valve may be incompetent, flow vortices may continue to form at the existing valve to help prevent stagnation of the blood at the base of the existing valve. Thus, the method of the present invention, by virtue of the placement of the prosthetic valve at the predetermined distance, will allow retrograde flow vortices to continue to form at the existing valve.

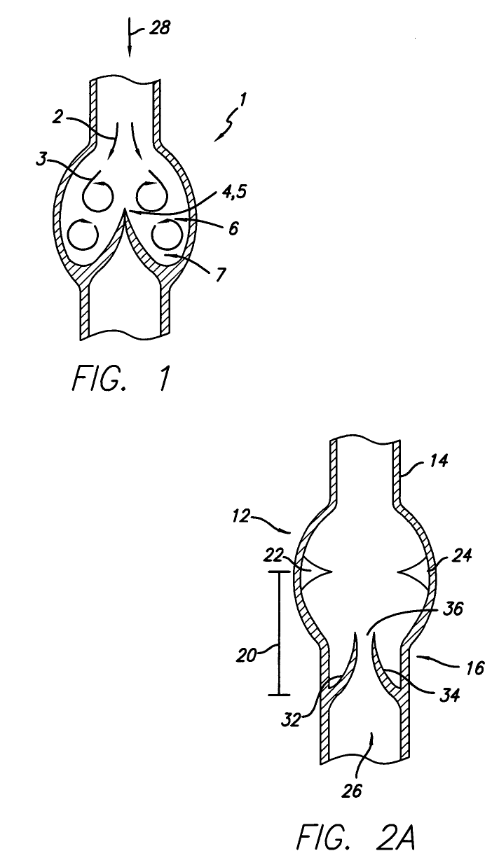

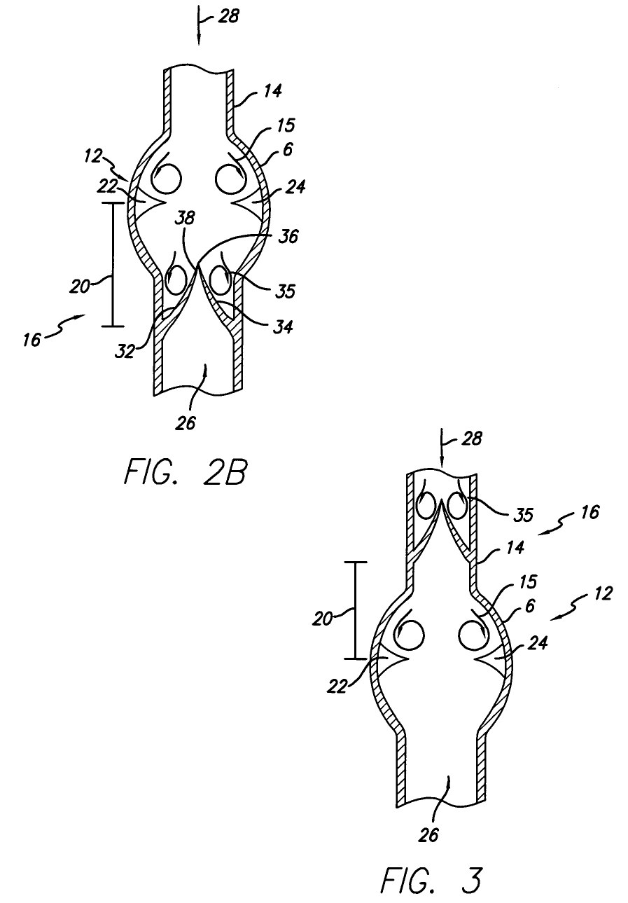

An embodiment of the present invention is illustrated in FIGS. 2A and 2B. An incompetent valve 12 is shown in the vessel 14 having a prosthetic valve device 16 positioned at a distance 20 away from the incompetent valve 12. As shown, the leaflets 22, 24 of the incompetent valve 12 extend into the vessel 14. With an incompetent existing valve 12, fluid can flow through the valve in a first direction 26 and also in a second direction 28 back through the valve 12. When the existing valve 12 is competent, the leaflets 22, 24 open to allow fluid to flow in the first direction 26, but contact each other to substantially prevent retrograde flow back through the valve 12 in the second direction 28 (as shown in FIG. 1). As shown in FIGS. 2A and 2B, when the existing valve 12 is incompetent, the leaflets 22, 24 fail to contact each other to form a contact surface and thus fail to substantially prevent fluid flow through the valve 12 in the second direction 28. The incompetent leaflets 22, 24 may partially extend into the vessel 14 and affect the blood flow in the second direction 28 to some extent. As described above, the existing valve 12 includes rounded sinuses 6 that may facilitate creation of flow vortices 15 together with the leaflets 22, 24 even in the incompetent valve 12 where the leaflets 22, 24 fail to contact each other.

As shown in FIGS. 2A and 2B, the prosthetic valve device 16 is positioned at a distance 20 from the existing valve 12. The valve device 16 may include one or more leaflets defining an opening 36 in the prosthetic valve 16 when the fluid flow is in the first direction 26 (FIG. 2A). As shown in FIG. 2B, leaflets 32, 34 extend into the vessel 14 and form a contact surface 38. One of skill in the art will understand that the valve device 16 may include one leaflet, or a plurality of leaflets, e.g. two, three, four, five or more leaflets, within the scope of the present invention. The leaflets 32 and 34 may be formed with a flexible material and move outwardly to form the opening 36 when subjected to fluid flow in the first direction 26 in the vessel 14 (FIG. 2A) and move inwardly to substantially close the opening 36 and form the contact surface 38 when subjected to fluid flow in the second direction 28 as shown in FIG. 2B. Positioning of the valve device 16 within the vessel 14 with respect to the incompetent valve 12 at the distance 20 may be important for the functional aspects of the valve device 16 to work properly or more effectively. Positioning of the valve device 16 at the distance 20 also affects aspects of the valve 12. Preferably, placement of the valve device 16 at the distance 20 allows flow vortices 15 to form at the valve 12. Additional flow vortices 35 may be formed at the valve 16.

As described above, the prosthetic valve device 16 is positioned at a predetermined distance 20 away from the incompetent valve 12 in the vessel 14. The valve device 16 may be positioned above or, preferably below, the incompetent valve 12 in the vessel 14. As used herein, the term “below” refers to positioning of the valve 16 in the vessel 14 before the valve 12 with respect to the fluid flow in the first direction 26 so that the fluid in the vessel 14 contacts the prosthetic valve 16 and then the existing valve 12. For example, in the direction of flow towards the heart in a venous vessel (the first direction 26) the valve 16 is positioned below the valve 12 as shown in FIGS. 2A and 2B. FIG. 3 illustrates the valve 16 positioned above the valve 12 in the vessel 14 with respect to the first flow direction 26.

One of skill in the art will recognize that a plurality of distances between the prosthetic valve 16 and the incompetent valve 12, wherein the retrograde flow vortices continue to form at the incompetent valve 12, are possible within the scope of the present invention. The distance 20 may depend on several factors, including the diameter of the vessel and the valve and the distance between natural valves and any prosthetic valves and the amount of retrograde flow through the existing valve 12 in the second direction 28. As discussed above, the existing valve 12 may still affect the amount and force of flow through the valve 16. Preferably, the distance 20 between the existing valve 12 and the prosthetic valve 16 will help to alter the force of the flow on the prosthetic valve 16 and allow flow vortices 15 to form at the existing valve 12 and at the prosthetic valve 16. Placement of the valve 16 at the distance 20 from the existing valve 12 may also allow remodeling of the valve 16 by the native tissue without interference from the existing valve 12. For example, interference with flow vortices 15 and remodeling occurs when the valve 16 is placed directly at the site of the existing valve 12 rather than at a distance 20 from the existing valve 12. Preferably, the distance 20 between the prosthetic valve 16 and the existing valve 12 may be about 0.5 to about 25 mm, more preferably about 2 to about 15 mm, most preferably about 5 to about 10 mm. The valve 16 may be placed at the distance 20 above or below the valve 12.

Additional considerations for the selection of the distance 20 between the existing valve 12 and the prosthetic valve 16 may include factors affecting the flow through the vessel at the existing valve 12. The factors affecting flow include, but are not limited to, the taper of the vessel 14 leading up to or extending away from the valve 12, the existence of side branching vessels near the existing valve 12, the elliptical ratio near the existing valve, the extent of the incompetence of the existing valve 12, the amount of retrograde flow through the existing valve 12 and the vortices formed at the existing valve 12. Preferably, placement of the prosthetic valve 16 is at the distance 20 from the existing valve 12 in a vessel 14 where the tapering of the vessel 14 does not significantly affect the expansion of the prosthetic valve 16 when the valve 16 is placed in the vessel 14. For example, the valve 16, when positioned at the distance 20 from the existing valve 12 in a substantially non-tapering vessel 14, the valve 16 may radially expand generally uniformly along the length of the valve 16 when placed at the implantation position. One of skill in the art will understand that the valve 16 is placed in the vessel 14 wherein uneven expansion of the prosthetic valve 16 after delivery to the site is minimized, for example, avoiding placement where the taper of the vessel 14 would allow expansion of the first end, but would restrict expansion of the second end.

The prosthetic valve 16 may also be preferably placed at the distance 20 from the existing valve 12 where no side branching vessels extend from the vessel 14 to disrupt the flow between the prosthetic valve 16 and the existing valve 12. Alternatively, the prosthetic valve 16 may be placed at the distance 20 from the existing valve 12 when side branching vessels do extend from the vessel 14 where any disruption to the flow through the vessel 14 due to the side branching vessels is minimal and does not affect the flow vortices formed or any remodeling processes that may occur at the prosthetic valve 16.

Another factor for consideration for determination of the distance 20 includes the elliptical ratio of the vessel 14 into which the prosthetic valve 16 may be placed. An elliptical ratio of 100% refers to a perfect circle, and an elliptical ratio of <100% refers to an oval shaped vessel. Preferably, the valve 16 is placed at a distance 20 from the valve 12 in a vessel having an elliptical ratio of greater than 80%, more preferably greater than 90%. Positioning of the prosthetic valve 16 may also consider the orientation of the prosthetic valve 16 with respect to the existing valve 12. The orientation of the prosthetic valve 16 may also affect the fluid flow through the existing vessel 12. The considerations for selection of the distance 20 including factors affecting fluid flow such as tapering vessels, side branching vessels, the elliptical ratio, the extent of incompetence, the amount of retrograde flow, and the flow vortices formed at the existing valve 12 described above also apply to considerations for selecting the orientation.

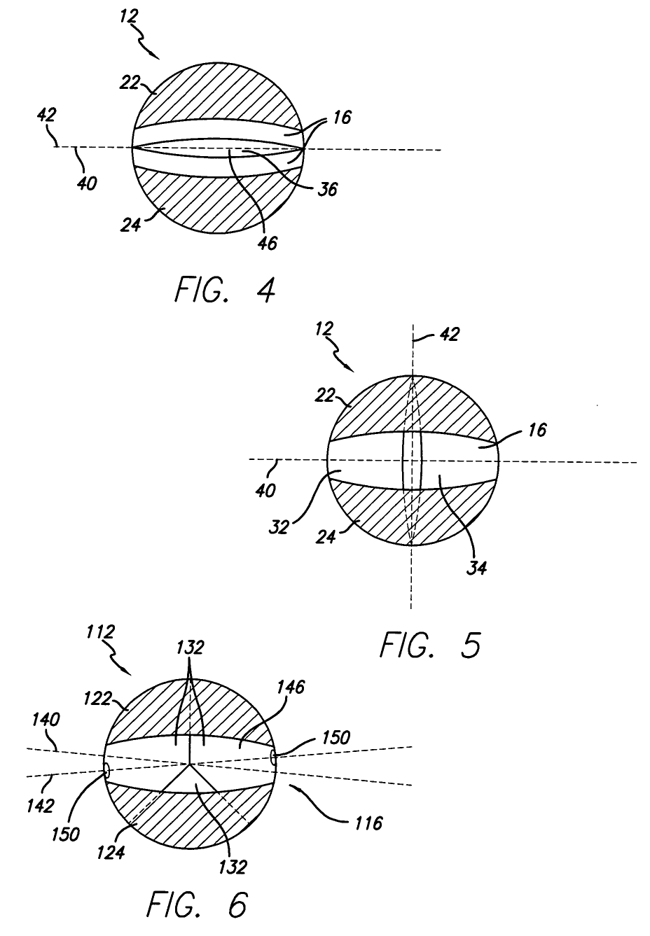

As shown in FIG. 4, the existing valve 12 includes an axis 40 along an opening 46 formed between the leaflets 22 and 24. The prosthetic valve 16 may be positioned in the vessel 14 with respect to the axis 40 of the existing valve 12. As shown in the cross-sectional view of FIG. 4, the prosthetic valve 16 may include an axis 42 along the opening 36. The opening 36 is formed when flow is in the first direction 26 through the leaflets 32 and 34, although the opening 36 may also be formed by movement of one or more leaflets. As shown in FIG. 4, the axis 42 of the prosthetic valve 16 may be defined by the leaflets 32 and 34 when a pair of leaflets is present and may be substantially aligned with the axis 40 of the existing valve 12. The prosthetic valve 16 in FIG. 4 is shown positioned below the existing valve 12 (as in FIG. 2), and fluid flows through the opening 36 of the prosthetic valve 16 and then the opening 46 of the existing valve 12 which openings 36, 46 are also substantially aligned. Alternatively, as discussed above and shown in FIG. 3, the prosthetic valve 16 may also be positioned above valve 12 and may have the axes 40 and 42 substantially aligned. The axis 42 of the prosthetic valve 16 may also be determined using imageable elements (discussed below). For example, when an uneven number of leaflets are included in the valve 16, imageable elements may be placed on the valve to define the axis 42.

Additional orientations of the axis 42 of prosthetic valve 16 with respect to the axis 40 of the existing valve 12 are possible. As shown in FIG. 5, the axis 40 of the prosthetic valve 16 may be offset with respect to the axis 40 of the existing valve 12. Any degree of rotation of the axis 42 from the axis 40 is possible within the scope of the present invention. For example, the prosthetic valve 16 may be positioned within the vessel 14 so that the axis 42 of the valve 16 is offset from the axis 40 of the valve 12 preferably by about 1 degree to about 179 degrees, more preferably from about 45 degrees to about 120 degrees, most preferably from about 80 degrees to about 110 degrees. As shown in FIG. 5, the axis 42 of the valve 16 is offset from the axis 40 of the valve 12 by about 90 degrees. The valve 16 is shown positioned in the vessel 14 below the valve 12 with respect to the direction 26 of flow through the valves 12 and 16 (see FIG. 2). Alternatively, as described above and shown in FIG. 3, the prosthetic valve 16 may be positioned in the vessel 14 above the existing valve 12 with respect to the direction 26 of flow through the valves 12 and 16 and be aligned such that the axis 42 of the prosthetic valve 16 is offset from the axis 40 of the valve 12.

An alternative embodiment of the present invention is shown in FIG. 6 where a prosthetic valve 116 is positioned below the existing valve 112. The prosthetic valve 116 includes three leaflets 132 shown in a closed configuration below the opening 146 in the existing valve 112. The prosthetic valve 116 includes imageable elements 150 that may be used for identifying an axis 142 of the prosthetic valve and for positioning the valve device 116 with respect to the axis 140 of the existing valve 112 and for determining the distance between the valve 116 and the valve 112 in the vessel 14 as described above. As shown in FIG. 6, the axis 142 of the prosthetic valve 116 is offset from the axis 140 of the existing valve 112. Similar to the embodiments described above, the prosthetic valve 116 may be positioned in the vessel 14 above or below the existing valve 112. The axis 142 of the prosthetic valve 116 may be in any position with respect to the axis 140.

As described above, the prosthetic valve 16, and similarly, the valve 116, is positioned at a distance 20 from the existing valve 12, and optionally, the prosthetic valve 16 may also be positioned with the axis 42 orientated with respect to the axis 40 of the valve 12. The selected position and the orientation of the valve 16 with respect to the valve 12 will depend on vessel diameter, the rate of flow through the vessel 14, the extent of incompetence of the valve 12 and other factors, including, but not limited to, the factors affecting flow through the vessel 14, discussed above, as will be understood by one of skill in the art.

Determination of the position for placement of the prosthetic valve 16 at the distance 20 from the existing valve 12 begins with detection of the existing, defective valve 12. The valve 12 may be detected using any method known to one of skill in the art, including, but not limited to, contrast and magnetic resonance venography, Doppler ultrasound, such as triplex ultrasound and continuous wave Doppler, and ambulatory strain gauge plethysmography. For example, the location of the defective existing valve 12 may be determined using direct contrast venography by injecting contrast dye into a patient's vein, such as the greater saphenous vein, near the groin and visualizing the contrast injection fluoroscopically. The valve 12 may be identified by a bulge in the vessel indicating a sinus formed at the valve 12. When the patient is in an upright position, visualization of the dye below the sinus demonstrates the position of a defective valve. If the valve is functioning properly, little or no contrast dye will be visible below the valve.

Another method for detecting the defective valve 12 uses Doppler ultrasound to measure blood flow. For example, evaluation of the superficial venous system may be conducted using a linear 7.5 to 10 MHz tranducer capable of displaying grayscale or color two-dimensional and pulse-wave Doppler images. The patient is examined in the standing position beginning at the saphenofemoral junction. The greater saphenous vein is followed from its junction beyond the level of any visible varicose veins. The caliber of the greater saphenous vein is assessed. Normally, the vein is ≦4 mm in diameter. Veins having a diameter greater than 7 mm have a high incidence of reflux. Segments having a diameter greater than 5 mm or having varicose segments may then be evaluated with pulse-wave Doppler to look for antegrade flow followed by retrograde flow after a quick, firm compression of a peripheral segment of the greater saphenous vein. A defective valve is identified by retrograde flow of ≧0.5 seconds. Additional venous segments may be measured using the same methods. (See Min et al., J. Vasc. Interv. Radiol. 2003: 14:1233-1241; van Bemmelen, et al., J. Vasc. Surg. 1989: 10:425-31.)

Any type of valve device known to one of skill in the art may be used with the methods of the present invention. Examples of suitable valve devices include, but are not limited to, the devices described in U.S. Pat. No. 6,508,833 to Pavcnik for a MULTIPLE SIDED INTRALUMINAL MEDICAL DEVICE, U.S. Publication Nos. 2001/0039450 and 2004/0186558 to Pavcnik for an IMPLANTABLE VASCULAR DEVICE, PCT publication WO 2004/096100 A1 to Case et al. for an ARTIFICIAL VALVE PROSTHESIS WITH IMPROVED FLOW DYNAMICS, and U.S. Publication No. 2004/0049262 to Obermiller et al. for STENT VALVES AND USES OF SAME, each of which is hereby incorporated by reference in its entirety. Additional implantable devices may be used with the methods of the present invention, including any type of expandable intraluminal device, non-expandable intraluminal devices, and conventional stents.

The prosthetic valve 16 may include one or more imageable elements located on the prosthetic valve 16 that are configured to facilitate placement of the valve 16 in the vessel 14 at the desired distance from the existing valve 12, in the desired orientation with respect to the axis 40 of the existing valve 12, or both. Imaging using an external imageable element may also be used for placing the valve 16 at the desired distance from the existing valve 12. The imageable elements may be viewed by devices such as a fluoroscope, X-ray, ultrasound, M.R.I., and others known to one of skill in the art. For example, the position of the existing valve 12 may be determined using the techniques described above. The position of the valve 12 may then be identified using an external measurement system placed beneath the patient's vessel 14 where the markers are visible by an external imaging device. The markers indicating the position of the valve 12 may be imaged while the prosthetic valve 16 is being positioned within the vessel 14 to place the valve 16 at the desired distance 20 from the valve 12 and in the desired orientation as will be understood by one skilled in the art.

The prosthetic valve 16, 116 itself may include an imageable element 50, 150 such as shown in FIGS. 6 and 7. Preferably, the imageable element 50, 150 is configured on the prosthetic valve 16, 116 to allow the clinician to adjust the position of the valve 16, 116 within the vessel 14 for placement in the desired orientation or distance 20 from the existing valve 12, 112. For example, the imageable element 50 may be placed at the opening 36, such as shown in FIG. 7, or on a structural feature of the valve 116 such as an anchoring means, covering, leaflet, frame, or others, for example as shown in FIG. 6. The imageable element 50, 150 will allow the clinician to position the valve 16, 116 in the vessel 14 in the desired orientation in the delivery device during implantation and monitor the position of the valve 16, 116 after implantation. Preferably, the imageable element 50, 150 allows the clinician to determine the axis 42, 142 of the valve 16, 116 to position the axis 42, 142 in the vessel 14 with respect to the axis 40, 140 of the existing valve 12, 112. As shown in FIG. 6, two imageable elements 50 are placed at the opening 36 on the leaflets 32 and 34. The imagable elements may be elongate to show the axis 42 of the prosthetic valve 16 under fluoroscopic guidance, for example, when the imageable elements 50 are formed from gold radiopaque material. A single imageable element 50 on either the leaflet 32 or 34 may also be used. The imageable element 50 may be located on other portions of the valve 16, such as on the structure and still allow the clinician to orient the axis 42 of the prosthetic valve 16 within the vessel 14. Similarly, a pair of imageable elements 150 may be used to determine the axis 140 of the prosthetic valve 116 to orient the axis 142 with respect to the axis 140 of the existing valve 112.

The imageable element 50, 150 may be applied to the prosthetic valve 16, 116 by any well known technique, including but not limited to, dipping, electrostatic deposition, spraying, painting, overlaying, wrapping and others. For example, a portion of the prosthetic valve 16, 116 may be dipped in molten gold. Optionally, a protective polymer overcoat may be applied to prevent degradation of the imaging material. A polymer resin coating may be applied to a portion of the valve 16, 116 that includes radiopaque filler material such as barium sulfate, bismuth, or tungsten powder. Alternatively, the imageable element 50, 150 may be formed from radiopaque wire or thread including gold, platinum, titanium and the like that may be used to form a portion of the prosthetic valve 16, 116. Preferably, the imageable element 50, 150 will not alter or interfere with the function of the valve 16, 116. Exemplary prosthetic valve devices and imageable elements are further described in U.S. Publication No. 2004/0167619, which is incorporated by reference herein in its entirety.

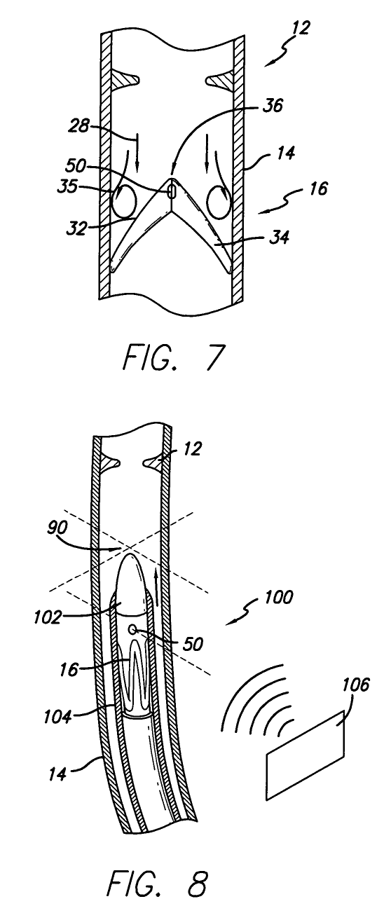

The prosthetic valve 16 may be delivered to the desired position within the vessel 14 with respect to the existing valve 12 using a delivery device such as a delivery catheter. Any delivery device known to one of skill in the art may be used for delivering the prosthetic valve 16, 116 to the position within the vessel 14 and will be described with respect to the prosthetic valve 16. An exemplary delivery device 100 is shown in FIG. 8 for placing the prosthetic valve 16 at position 90 in the vessel 14. The delivery device 100 enters the vessel 14 through an incision (not shown) made in the vessel below the implantation site 90, and the delivery device 100 is advanced toward the heart. Alternatively, the incision in the vessel 14 may be made above the implantation site 90 and the delivery device 100 advanced away from the heart for positioning the prosthetic valve 16. Typically, the delivery device 100 includes an inner member 102 on which the valve 16 is mounted and constrained within a delivery sheath 104. As shown in FIG. 7, the prosthetic valve 16 includes an imageable element 50 that may be imaged with an external imaging device 106 for positioning the valve 16 at a selected position at the distance 20 from the existing valve 12 within the vessel 14 and/or in a selected orientation. The prosthetic valve 16 is delivered to the selected position in the vessel 14 using the delivery device 100. The prosthetic valve 16 is deployed and implanted at the selected position and the delivery device 100 is removed from the vessel 14. Exemplary delivery devices suitable for implanting the valve 16 include U.S. Publication Nos. 2004/0225344 to Hoffa et al. for DEVICES, KITS AND METHODS FOR PLACING MULTIPLE INTRALUMINAL MEDICAL DEVIES IN A BODY and 2003/0144670 to Pavcnik et al. for a MEDICAL DEVICE DELIVERY SYSTEM, which are incorporated by reference herein in their entirety. Alternatively, rapid exchange catheters may be used for delivery of the valve device to the desired position, such as a rapid exchange delivery balloon catheter which allows exchange from a balloon angioplasty catheter to a delivery catheter without the need to replace the angioplasty catheter wire guide with an exchange-length wire guide before exchanging the catheters. Exemplary rapid exchange catheters that may be used to deliver the valve device of the present invention are described in U.S. Pat. Nos. 5,690,642; 5,814,061; and 6,371,961 which are herein incorporated by reference in their entirety.

Although the invention herein has been described in connection with a preferred embodiment thereof, it will be appreciated by those skilled in the art that additions, modifications, substitutions, and deletions not specifically described may be made without departing from the spirit and scope of the invention as defined in the appended claims. The scope of the invention is defined by the appended claims, and all devices that come within the meaning of the claims, either literally or by equivalence, are intended to be embraced therein.

1. A method of implanting a prosthetic valve in a body vessel having fluid flow therethrough, the method comprising:
identifying a position of an existing valve in the body vessel;
determining one or more factors affecting fluid flow in at least one of a first direction and a second direction at the existing valve; the factor being selected from the group consisting of: determining an affect of side branching vessels, determining an elliptical ratio of the body vessel, determining a flow velocity through the body vessel, determining a taper in a diameter of the body vessel, and determining an amount of fluid flow through the existing valve in the second direction;
selecting an implant position for a prosthetic valve at a distance away from the existing valve position in consideration of the factor;
providing the prosthetic valve for delivery to the implantation position, the prosthetic valve having at least one flexible member movable between a first position that permits fluid flow in a first direction and a second position that substantially prevents fluid flow in a second direction;
delivering the prosthetic valve to the implantation position; and
implanting the prosthetic valve at the implantation position.

2. The method of claim 1 wherein determining the factor comprises determining an affect of side branching vessels.

3. The method of claim 1 wherein determining the factor comprises determining an elliptical ratio of the body vessel.

4. The method of claim 1 wherein determining the factor comprises determining a taper in a diameter of the body vessel.

5. The method of claim 1 wherein determining the factor comprises determining a flow velocity through the body vessel.

6. The method of claim 1 wherein determining the factor comprises determining an amount of fluid flow through the existing valve in the second direction.

7. The method of claim 1 wherein the distance is about 0.5 millimeter to about 25 millimeters away from the existing valve.

8. The method of claim 1 wherein the distance is about 2 millimeters to about 15 millimeters away from the existing valve.

9. The method of claim 1 wherein the distance is about 5 millimeters to about 10 millimeters away from the existing valve.

10. The method of claim 1 wherein the position for implantation is below the existing valve with respect to the first flow direction.

11. The method of claim 1 wherein the position for implantation is above the existing valve with respect to the first flow direction.

12. The method of claim 1 comprising determining the implantation position to substantially maintain flow vortices at the existing valve.

13. The method of claim 1 further comprising providing an imageable element on the prosthetic valve for visualizing the prosthetic valve in the body vessel.

14. A method for modulating fluid flow through a body vessel, the method comprising:
identifying an existing incompetent valve in the body vessel, the existing valve allowing fluid flow therethrough in a first direction and a second direction;
determining an implantation site for placement of a prosthetic valve at a distance away from the existing valve based on one or more factors affecting fluid flow in at least one of a first direction and a second direction at the existing valve; the factor being selected from the group consisting of: determining an affect of side branching vessels, determining an elliptical ratio of the body vessel, determining a flow velocity through the body vessel, determining a taper in a diameter of the body vessel, and determining an amount of fluid flow through the existing valve in the second direction;
providing the prosthetic valve having at least one flexible member movable between a first position that permits fluid flow in the first direction and a second position that substantially prevents fluid flow in the second direction;
delivering the prosthetic valve to the implantation site; and
implanting the prosthetic valve at the implantation site;
wherein implanting the prosthetic valve at the distance modifies fluid flow through the body vessel by substantially preventing fluid flow in the second direction.

15. The method of claim 14 wherein the distance is about 2 millimeters to about 15 millimeters away from the existing valve.

16. The method of claim 14 wherein the distance is about 5 millimeters to about 10 millimeters away from the existing valve.

17. The method of claim 14 wherein the position for implantation is below the existing valve with respect to the first flow direction.

18. A method of implanting a prosthetic valve in a body vessel having a fluid flow therethrough, the method comprising:
identifying a first axis of an opening in an existing valve in the body vessel;
determining at least one factor affecting fluid flow in at least one of the first direction and the second direction at the existing valve;
selecting an implantation position at a distance away from the existing valve in consideration of the at least one factor;
providing the prosthetic valve comprising an opening having a second axis;
delivering the prosthetic valve to the implantation position in the body vessel;
positioning the second axis with respect to the first axis; and
implanting the prosthetic valve in the body vessel with the second axis positioned with respect to the first axis and at the distance away from the existing valve.

19. The method of claim 18 wherein the first axis and the second axis are aligned.

20. The method of claim 18 wherein the first axis and the second axis are offset with respect to each other.
